(19) United States
(12) Patent Application Publication
(10) Pub. No.: US 2022/0398522 A1
(43) Pub. Date: Dec. 15, 2022

(54) MEDICAL FACILITY EVALUATION APPARATUS, MEDICAL FACILITY EVALUATION METHOD, AND COMPUTER PROGRAM

(71) Applicant: NEC Corporation, Minato-ku, Tokyo (JP)

(72) Inventors: Masahiro HAYASHITANI, Tokyo (JP); Masahiro KUBO, Tokyo (JP)

(73) Assignee: NEC Corporation, Minato-ku, Tokyo (JP)

(21) Appl. No.: 17/771,909

(22) PCT Filed: Nov. 1, 2019
(86) PCT No.: PCT/JP2019/043101

§ 371 Date: Apr. 26, 2022

(51) Int. Cl. G06Q 10/06; G16H 40/20

(52) U.S. Cl. CPC G06Q 10/06393; G16H 40/20

(57) ABSTRACT

A medical facility evaluation apparatus includes: an acquisition unit that obtains a quantitatively measured evaluation information about a medical facility that is a candidate of a transfer destination of a patient; and a calculation unit that calculates a score indicating goodness of fit of the medical facility as the transfer destination with respect to the patient, on the basis of the evaluation information. According to this medical facility evaluation apparatus, scoring based on the quantitatively measured evaluation information makes it possible to find a medical facility with high goodness of fit as a transfer destination of a patient.

### TECHNICAL FIELD

The present invention relates to a medical facility evaluation apparatus, a medical facility evaluation method, and a computer program that evaluate a medical facility such as a hospital.

### BACKGROUND ART

A known technique/technology of this type is a technique/technology of searching for a medical facility suitable for a patient. For example, Patent Literature 1 discloses a technique/technology of searching for a long-term care hospital located in a region desired by a patient. Patent Literature 2 discloses a technique/technology of searching for a hospital to which a patient of a general hospital is transferred after an initial treatment. Patent Literature 3 discloses a technique/technology of extracting and presenting candidates of hospitals with a high degree of user satisfaction.

### CITATION LIST

### Patent Literature

### Patent Literature 1: JP2008-129620A

### Patent Literature 2: JP2017-194790A

### Patent Literature 3: JP2018-195164A

### SUMMARY

### Technical Problem

In order to find a medical facility suitable for a patient, it is preferable to consider an evaluation for the medical facility (e.g., an evaluation by a user, an evaluation by a staff, an evaluation based on facility data, etc.). In the techniques/technologies described in Patent Literatures 1 to 3, however, a quantitative evaluation of a medical facility is not conducted. Therefore, there is a technical problem that it is hard to find a medical facility suitable for a patient.

The present invention has been made in view of the above-described problems, and it is an example object of the present invention to provide a medical facility evaluation apparatus, a medical facility evaluation method, and a computer program that are configured to find a medical facility with high goodness of fit as a transfer destination of a patient.

### Solution to Problem

A medical facility evaluation apparatus according to an example aspect of the present invention includes: an acquisition unit that obtains a quantitatively measured evaluation information about a medical facility that is a candidate of a transfer destination of a patient; and a calculation unit that calculates a score indicating goodness of fit of the medical facility as the transfer destination with respect to the patient, on the basis of the evaluation information.

A medical facility evaluation method according to an example aspect of the present invention includes: an acquisition step of obtaining a quantitatively measured evaluation information about a medical facility that is a candidate of a transfer destination of a patient; and a calculation step of calculating a score indicating goodness of fit of the medical facility as the transfer destination with respect to the patient, on the basis of the evaluation information.

A computer program according to an example aspect of the present invention allows a computer to execute: an acquisition step of obtaining a quantitatively measured evaluation information about a medical facility that is a candidate of a transfer destination of a patient; and a calculation step of calculating a score indicating goodness of fit of the medical facility as the transfer destination with respect to the patient, on the basis of the evaluation information.

### Effect of the Invention

According to the medical facility evaluation apparatus, the medical facility evaluation method, and the computer program in the respective aspects described above, scoring based on a quantitative evaluation information makes it possible to find a medical facility with high goodness of fit as a transfer destination of a patient.

### BRIEF DESCRIPTION OF DRAWINGS

FIG. 1 is a block diagram illustrating an overall configuration of a medical facility evaluation apparatus according to a first example embodiment.

FIG. 2 is a block diagram illustrating a hardware configuration of the medical facility evaluation apparatus according to the first example embodiment.

FIG. 3 is a flowchart illustrating a flow of operation of the medical facility evaluation apparatus according to the first example embodiment.

FIG. 4 is a diagram illustrating an example of a score calculation method using a weight.

FIG. 5 is a block diagram illustrating an overall configuration of the medical facility evaluation apparatus according to a second example embodiment.

FIG. 6 is a flowchart illustrating a flow of operation of the medical facility evaluation apparatus according to the second example embodiment.

FIG. 7 is a diagram illustrating a display example of a transfer destination suited for a patient.

### DESCRIPTION OF EXAMPLE EMBODIMENTS

A medical facility evaluation apparatus, a medical facility evaluation method, and a computer program according to example embodiments will be described below with reference to the drawings.

### First Example Embodiment

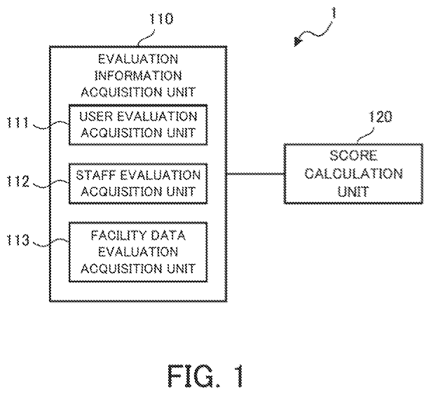

A medical facility evaluation apparatus according to a first example embodiment will be described with reference to FIG. 1 to FIG. 4.

### (Apparatus Configuration)

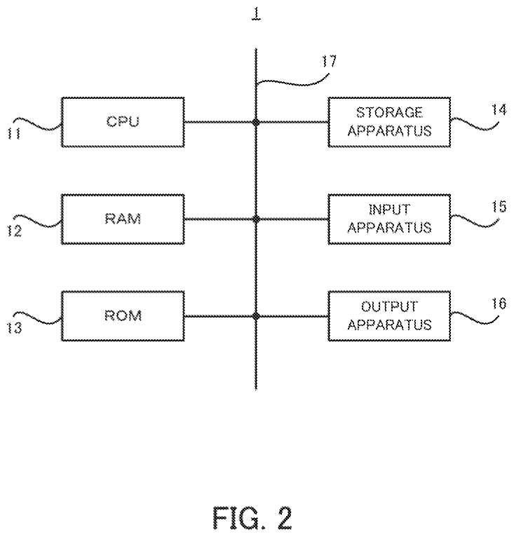

First, a configuration of the medical facility evaluation apparatus according to the first example embodiment will be described with reference to FIG. 1 and FIG. 2. FIG. 1 is a block diagram illustrating an overall configuration of the medical facility evaluation apparatus according to the first example embodiment. FIG. 2 is a block diagram illustrating a hardware configuration of the medical facility evaluation apparatus according to the first example embodiment.

In FIG. 1, a medical facility evaluation apparatus 1 according to the first example embodiment is an apparatus that evaluates a medical facility that is a transfer destination of a patient. The term “medical facility” here is a broad concept that includes a wide variety of large and small hospitals such as an acute care hospital and a convalescent hospital, as well as a facility at which direct medical actions are not performed such as a nursing care facility, a nursing home, and a small-scale multifunctional facility. Furthermore, the “hospital transfer” in this example embodiment is assumedly moving from an acute care hospital to another hospital (e.g., a convalescent hospital, a nursing care facility, a small-scale multifunctional facility, etc.) in accordance with a patient's symptom. The medical facility evaluation apparatus 1 includes an evaluation information acquisition unit 110 and a score calculation unit 120 as main components.

The evaluation information acquisition unit 110 is configured to obtain an evaluation information about a medical facility. Here, the “evaluation information” is information about the evaluation of a medical facility and is information quantitatively measured. The evaluation information may be represented, for example, as an evaluation value indicating how high the evaluation is. Furthermore, the evaluation information acquisition unit 110 may be configured to obtain a plurality of evaluation informations (more specifically, an evaluation information about a plurality of different items). The evaluation information acquisition unit 110 according to this example embodiment includes a user evaluation acquisition unit 111, a staff evaluation acquisition unit 112, and a facility data evaluation acquisition unit 113 in order to obtain the plurality of evaluation informations.

The user evaluation acquisition unit 111 is configured to obtain a user evaluation information indicating an evaluation from a user of a medical facility (e.g., a patient and his/her family members, etc.). The user evaluation information includes, for example, information about a degree of user satisfaction. A method for quantitatively measuring the degree of user satisfaction will be described in detail later.

The staff evaluation acquisition unit 112 is configured to obtain a staff evaluation information indicating an evaluation from an outsourcer staff of of the medical facility. The outsourcer staff of the medical facility includes, for example, a nurse and a social worker who coordinate the hospital transfer at a transfer-source medical facility. The staff evaluation information is information obtained as a result of evaluating the medical facility that is the transfer destination, from a viewpoint of the staff of the outsourcer that transfers a patient (in other words, from a viewpoint of a transfer-source hospital side), and includes, for example, information evaluated from a coverage ratio of an acceptance condition of the patient who is transferred, a response to questions from the outsourcer, and the like.

The facility data evaluation acquisition unit 113 is configured to obtain a facility data evaluation information indicating an evaluation based on facility data on the medical facility. The facility data include data on past results, such as, for example, the number of days of hospital stay of a patient in the medical facility, the number of days from the start of transfer adjustment to the actual transfer, a recovery degree of the patient after transfer (e.g., FIM: Functional Independence Measure, etc.), and the number of patients transferred from the outsourcer in the past. In addition to those data, the facility data may include information about location, such as a distance from the outsourcer (in other words, a distance traveled at the time of transfer), means of transport from the outsourcer (e.g., public transportation such as trains, buses, etc.), and the presence of a trunk road, or information about a medical device and various equipment owned by the facility.

The score calculation unit 120 is configured to calculate a score of the medical facility on the basis of the evaluation information obtained by the evaluation information acquisition unit 110. The score calculated here expresses goodness of fit of the patient who is transferred, to the medical facility in a form of numerical values, etc. The goodness of fit indicates how suitable each medical facility is for the transfer of a certain patient. The score calculation unit 120 according to this example embodiment calculates one score for the medical facility, from the user evaluation information obtained by the user evaluation acquisition unit 111, the staff evaluation information obtained by the staff evaluation acquisition unit 112, and the facility data evaluation information obtained by the facility data evaluation acquisition unit 113. A specific method of calculating the score will be described in detail later.

As illustrated in FIG. 2, the medical facility evaluation apparatus 1 according to this example embodiment includes a CPU (Central Processing Unit) 11, a RAM (Random Access Memory) 12, a ROM (Read Only Memory) 13, and a storage apparatus 14. The medical facility evaluation apparatus 1 may also include an input apparatus 15 and an output apparatus 16. The CPU 11, the RAM 12, the ROM 13, the storage apparatus 14, the input apparatus 15, and the output apparatus 16 are connected through a data bus 17.

The CPU 11 reads a computer program. For example, the CPU 11 may read a computer program stored by at least one of the RAM 12, the ROM 13 and the storage apparatus 14. For example, the CPU 11 may read a computer program stored by a computer readable recording medium, by using a not-illustrated recording medium read apparatus. The CPU 11 may obtain (i.e., read) a computer program from a not-illustrated apparatus located outside of the medical facility evaluation apparatus 1, through a network interface. The CPU 11 controls the RAM 12, the storage apparatus 14, the input apparatus 15, and the output apparatus 16 by executing the read computer program. Especially in this example embodiment, when CPU 11 executes the read computer program, a functional block for evaluating the medical facility is implemented in the CPU 11. The evaluation information acquisition unit 110 and the score calculation unit 120 described above are implemented, for example, in this CPU 11.

The RAM 12 temporarily stores the computer program to be executed by the CPU 11. The RAM 12 temporarily stores the data that is temporarily used by the CPU 11 when the CPU 11 executes the computer program. The RAM 12 may be, for example, a D-RAM (Dynamic RAM).

The ROM 13 stores fixed data that is not normally changed. The fixed data may include the computer program to be executed by the CPU 11. The ROM 13 may be, for example, a P-ROM(Programmable ROM).

The storage apparatus 14 stores the data that is stored for a long term by the medical facility evaluation apparatus 1. The storage apparatus 14 may store the computer program to be executed by the CPU 11. The storage apparatus 14 may also operate as a temporary storage apparatus of the CPU 11. The storage apparatus 14 may include, for example, at least one of a hard disk apparatus, a magneto-optical disk apparatus, an SSD (Solid State Drive), and a disk array apparatus.

The input apparatus 15 is an apparatus that receives an input instruction from the user of the medical facility evaluation apparatus 1. The input apparatus 15 may include, for example, at least one of a keyboard, a mouse, and a touch panel.

The output apparatus 16 is an apparatus that outputs information about the medical facility evaluation apparatus 1 to the outside. For example, the output apparatus 16 may be a display apparatus that is configured to display the information about the medical facility evaluation apparatus 1.

Each of the input apparatus 15 and the output apparatus 16 may be a smart phone, a tablet, a PC, or the like used by medical personnel.

### (Description of Operation)

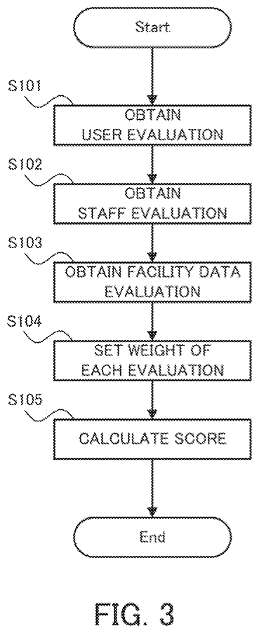

Next, with reference to FIG. 3, a flow of operation of the medical facility evaluation apparatus 1 according to the first example embodiment will be described. FIG. 3 is a flow chart illustrating the flow of the operation of the medical facility evaluation apparatus according to the first example embodiment.

As illustrated in FIG. 3, in operation of the medical facility evaluation apparatus 1 according to the first example embodiment, firstly, the user evaluation acquisition unit 111 obtains the user evaluation information (step S101). The staff evaluation acquisition unit 112 then obtains the staff evaluation information (step S102). The facility data evaluation acquisition unit 113 then obtains the facility data evaluation information (step S103). Incidentally, the step S101 to the step S103 are not limited to the order described above; they may be performed simultaneously in parallel, or may be performed before or after each other.

When each evaluation information is obtained, the score calculation unit 120 sets a weight to be used for calculating the score (i.e., a weight applied to each of the user evaluation information, the staff evaluation information, and the facility data evaluation information) (step S104). The score calculation unit 120 then uses the set weights to calculate the score of the medical facility (step S105).

### (Score Calculation Method)

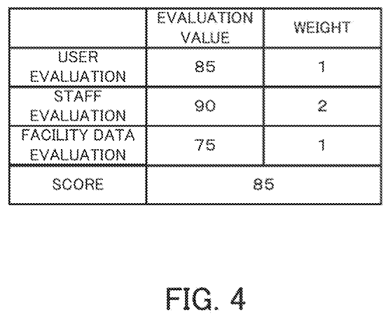

Next, with reference to FIG. 4, a specific score calculation method performed by the score calculation unit 120 (i.e., the details of the step S104 and the step S105 in FIG. 3) will be described. FIG. 4 is a diagram illustrating an example of the score calculation method using the weight.

In the example illustrated in FIG. 4, evaluation values “85”, “90”, and “75” are obtained as the user evaluation information, the staff evaluation information, and the facility data evaluation information. In this example, the evaluation value is assumed to be a maximum of 100 points. In addition, weights “1”, “2”, and “1” are respectively set for the user evaluation information, the staff evaluation information, and the facility data evaluation information. This weight may be set in advance in accordance with the importance of each evaluation information, or may be arbitrarily set at the time of score calculation.

The score calculation unit 120 calculates the score, for example, as a weighted average using the above evaluation values and weights. Specifically, the score calculation unit 120 calculates the score by using a calculation formula like the following equation (1)

(85×1+90×2+75×1)/4=85 (1)

As described above, the score calculated by the score calculation unit 120 is a value that takes into account the weights set in each evaluation information. In this way, it is possible to calculate the score that reflects the importance of each evaluation value. Note that the above-described score calculation method is merely an example, and the score may be calculated by using another method.

### (Quantification Method of Patient Satisfaction)

Next, a specific quantification method of patient satisfaction (i.e., a specific method of obtaining the user evaluation information) will be described.

The patient satisfaction may be determined in accordance with a time length required from when an inpatient calls the staff of the medical facility to when the staff of the medical facility responds. As a more specific example, the patient satisfaction is determined in accordance with a time length required from when an inpatient pushes a nurse call to when the staff of the medical facility arrives at the patient. In this case, as the time length required for the staff to start to respond becomes longer, the patient satisfaction may be determined to be high. The time length required for the staff to start to respond may be measured by using a wearable sensor, a GPS (Global Positioning System), an image captured by a camera, or a sensor installed on a bed or in the vicinity thereof.

Alternatively, the patient satisfaction may be determined in accordance with a cumulative time in which the staff of the medical facility responds to the inpatient. Specifically, as the cumulative time of the response of the staff becomes longer, the patient satisfaction may be determined to be high. The cumulative time of the response of the staff may be measured by using a wearable sensor, a GPS, an image captured by a camera, or the like.

Alternatively, the patient satisfaction may be obtained in accordance with an activity amount and sleeping hours of the inpatient. Specifically, as the activity amount of the patient increases, or as the sleeping hours of the patient becomes longer, the patient satisfaction may be determined to be high. The activity amount of the inpatient includes, for example, a rehabilitation amount. In addition, the activity amount of the inpatient may include a distance walked by the inpatient other than rehabilitation, and the amount of another activity performed by the inpatient. The activity amount and sleeping hours of the inpatient may be measured by using a wearable sensor or the like.

Alternatively, the patient satisfaction may be obtained in accordance with a conversation amount of the inpatient. Specifically, as the conversation amount of the patient increases, the patient satisfaction may be determined to be high. The conversation amount of the inpatient may be measured by using a voice recorder or the like.

Alternatively, the patient satisfaction may be obtained in accordance with a period in which an emotional state of the inpatient is positive (e.g., an emotion of “joy” or “pleasure”). Specifically, as the period in which the emotional state of the inpatient is positive (especially, a period in the later stage of hospitalization) becomes longer, the patient satisfaction may be determined to be high. The emotional state of the inpatient may be measured, for example, by using a camera image, an IoT device, a wearable sensor, or the like.

### Technical Effect

Next, a technical effect obtained by the medical facility evaluation apparatus 1 according to the first example embodiment will be described.

As described from FIG. 1 to FIG. 4, according to the medical facility evaluation apparatus 1 in the first example embodiment, the score of the medical facility (specifically, the score indicating how suitable each medical facility is for the transfer of a certain patient) can be preferably calculated from the quantitatively measured evaluation information. Therefore, it is possible to easily find an appropriate medical facility as the transfer destination, and to efficiently perform transfer adjustment (discharge adjustment).

### Second Example Embodiment

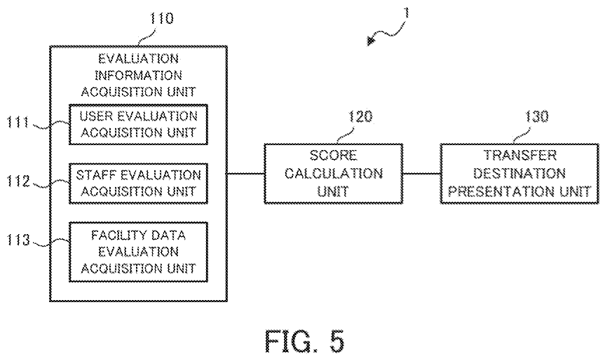

Next, a medical facility evaluation apparatus according to a second example embodiment will be described with reference to FIG. 5 to FIG. 7. The second example embodiment is partially different from the first example embodiment described above only in the configuration and operation, and is substantially the same in the other parts. For this reason, the parts that differ from the first example embodiment described above will be described below, and the other overlapping parts will not be described.

### (Apparatus Configuration)

Firstly, a configuration of the medical facility evaluation apparatus 1 according to the second example embodiment will be described with reference to FIG. 5. FIG. 5 is a block diagram illustrating an overall configuration of the medical facility evaluation apparatus according to the second example embodiment. Incidentally, in FIG. 5, the same components the those illustrated in FIG. 1 carry the same reference numerals.

As illustrated in FIG. 5, the medical facility evaluation apparatus 1 according to the second example embodiment includes a transfer destination presentation unit 130, in addition to the configuration in the first example embodiment (see FIG. 1).

The transfer destination presentation unit 130 is configured to present a medical facility suitable for transfer of the patient on the basis of a plurality of scores of medical facilities calculated by the score calculation unit. The transfer destination presentation unit 130 is configured, for example, to extract the medical facility suitable as the transfer destination from among a plurality of transfer destination candidates and to output a list of the extracted medical facilities to a display or the like.

### (Description of Operation)

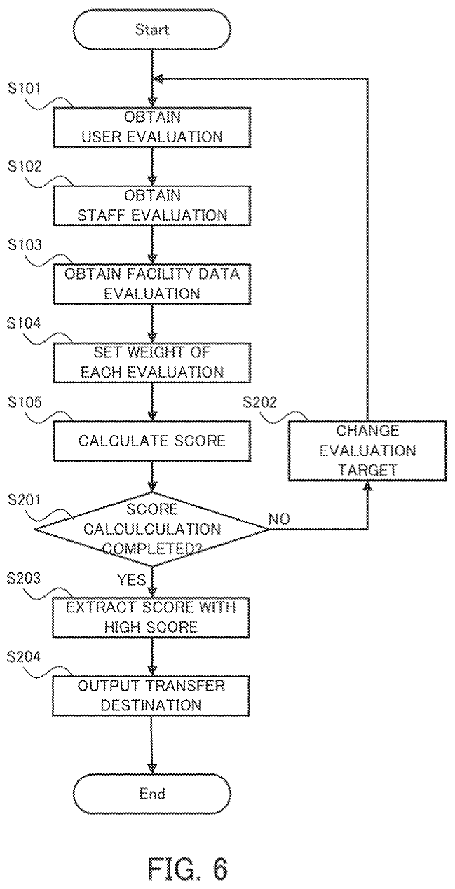

Next, with reference to FIG. 6, a flow of operation of the medical facility evaluation apparatus 1 according to the second example embodiment will be described. FIG. 6 is a flow chart illustrating the flow of the operation of the medical facility evaluation apparatus according to the second example embodiment. Incidentally, in FIG. 6, the same steps as those illustrated in FIG. 3 carry the same reference numerals.

As illustrated in FIG. 6, in operation of the medical facility evaluation apparatus 1 according to the second example embodiment, as in the first example embodiment, the evaluation information acquisition unit 110 obtains the respective evaluation informations (step S101 to S103), and the score calculation unit 120 calculates the score (step S104 to S105).

Then, in the second example embodiment, the transfer destination presentation unit 130 determines whether or not the scores of all the transfer destination candidates are calculated (step S201). That is, the transfer destination presentation unit 130 determines whether or not the score is calculated for all the medical facilities that can be selected by the patient as the transfer destination, or for some medical facilities that are selected in advance as the transfer destination.

If the score is not calculated for all the transfer destination candidates (the step S201:NO), an evaluation target is changed to a medical facility for which the score is not yet calculated (step S202), and the step S101 to the step S105 are performed. By repeating the process in this way, the scores of all the transfer destination candidates are calculated.

When the score is calculated for all the transfer destination candidates (the step S201: YES), the transfer destination presentation unit 130 extracts a high-score medical facility from the transfer destination candidates (step S203). The number of medical facilities extracted here may be only one with the highest score, or a plurality of medical facilities may be extracted in order from the top of the score. Alternatively, the number of medical facilities extracted may not be limited, and medical facilities with a score that is equal to or higher than a certain value may be extracted. Finally, the transfer destination presentation unit 130 outputs the extracted medical facility as the medical facility suitable for the transfer (step S204).

In the above-described example, the exemplified configuration is that the score is calculated for all the transfer destination candidates in the step S201, but it may be determined whether or not the score is calculated for a certain number or larger number of candidates among the transfer destination candidates. In this case, it is sufficient to extract the medical facilities with high scores from the transfer destination candidates for which the score is calculated.

In addition, when outputting the medical facility suitable for the transfer, it may be presented for each type of the medical facility. The medical facility that is the transfer destination may depend on the patient's symptom or the like; for example, a convalescent hospital is preferred as the transfer destination, while a nursing care facility or a multifunctional facility may not be suitable. In such a case, separate presentation in the convalescent hospital and other facilities makes it easier to select the medical facility suitable as the transfer destination.

### (Presentation of Transfer Destination)

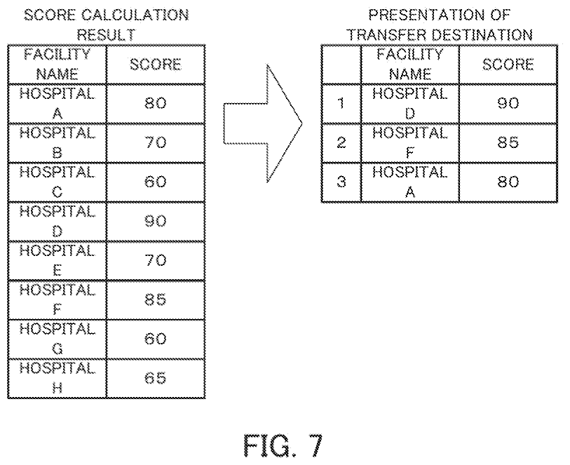

Next, with reference to FIG. 7, a specific example of the process of presenting the transfer destination suitable for the patient (i.e., the details of the step S203 and the step S204 in FIG. 6) will be described. FIG. 7 is a diagram illustrating a display example of the transfer destination suitable for the patient.

In the example illustrated in FIG. 7, the score is calculated for each of a plurality of transfer destination candidates (Hospitals A to H). Specifically, Hospital A has a score of “80”, Hospital B has a score of “70”, Hospital C has a score of “60”, Hospital D has a score of “90”, Hospital E has a score of “70”, Hospital F has a score of “85”, Hospital G has a score of “60”, and Hospital H has a score of “65”.

Here, it is assumed that the transfer destination presentation unit 130 is set to present three transfer destinations. In this case, the transfer destination presentation unit 130 presents “Hospital D (Score 90)” as the most suitable medical facility for the transfer, “Hospital F (Score 85)” as the second most suitable medical facility for the transfer, and “Hospital A (Score 80)” as the third most suitable medical facility for the transfer. In addition to the facility name and the score, data on each medical facility may be presented. Data on each medical facility includes, but is not limited to, for example, factors that cause a rise in the score for each medical facility (e.g., types and evaluation values of the evaluation information that contributes significantly to the rise in the score, etc.).

### Technical Effect

Next, a technical effect obtained by the medical facility evaluation apparatus 1 according to the second example embodiment will be described.

As described in FIG. 5 to FIG. 7, according to the medical facility evaluation apparatus 1 in the second example embodiment, the medical facility suitable for the transfer destination is presented on the basis of the scores of the transfer destination candidates. In this way, it is possible to determine the medical facility suitable for the transfer more easily than when only the score of each medical facility is outputted (i.e., presented).

### <Supplementary Notes>

With respect to the example embodiment described above, the following Supplementary Notes will be further disclosed.

### (Supplementary Note 1)

A medical facility evaluation apparatus described in Supplementary Note 1 is a medical facility evaluation apparatus including: an acquisition unit that obtains a quantitatively measured evaluation information about a medical facility that is a candidate of a transfer destination of a patient; and a calculation unit that calculates a score indicating goodness of fit of the medical facility as the transfer destination with respect to the patient, on the basis of the evaluation information.

### (Supplementary Note 2)

A medical facility evaluation apparatus described in Supplementary Note 2 is the medical facility evaluation apparatus described in Supplementary Note 1, further including a presentation unit that presents the medical facility to be recommended as the transfer destination of the patient on the basis of the score of each of a plurality of medical facilities.

### (Supplementary Note 3)

A medical facility evaluation apparatus described in Supplementary Note 3 is the medical facility evaluation apparatus described in Supplementary Note 2, wherein the presentation unit presents a factor that causes a rise in the score of the medical facility to be recommended as the transfer destination of the patient.

### (Supplementary Note 4)

A medical facility evaluation apparatus described in Supplementary Note 4 is the medical facility evaluation apparatus described in any one of Supplementary Notes 1 to 3, wherein the evaluation information includes at least one of an evaluation by a user of the medical facility, an evaluation by an outsourcer staff of the medical facility, and an evaluation based on information about the medical facility.

### (Supplementary Note 5)

A medical facility evaluation apparatus described in Supplementary Note 5 is the medical facility evaluation apparatus described in Supplementary Note 4, wherein the evaluation information includes information about a time length required from when an inpatient of the medical facility calls a staff of the medical facility to when the staff of the medical facility responds.

### (Supplementary Note 6)

A medical facility evaluation apparatus described in Supplementary Note 6 is the medical facility evaluation apparatus described in Supplementary Note 4 or 5, wherein the evaluation information includes information about a cumulative time in which a staff of the medical facility responds to an inpatient of the medical facility.

### (Supplementary Note 7)

A medical facility evaluation apparatus described in Supplementary Note 7 is the medical facility evaluation apparatus described in any one of Supplementary Notes 4 to 6, wherein the evaluation information includes at least one of information about an activity amount, sleeping hours, and a conversation amount of an inpatient of the medical facility.

### (Supplementary Note 8)

A medical facility evaluation apparatus described in Supplementary Note 8 is the medical facility evaluation apparatus described in any one of Supplementary Notes 4 to 7, wherein the evaluation information includes information about a period in which an emotional state of an inpatient of the medical facility is positive.

### (Supplementary Note 9)

A medical facility evaluation apparatus described in Supplementary Note 9 is the medical facility evaluation apparatus described in any one of Supplementary Notes 1 to 8, wherein the acquisition unit obtains a plurality of types of evaluation informations, and the calculation unit calculates the score by weighting each of the plurality of types of evaluation informations.

### (Supplementary Note 10)

A medical facility evaluation method described in Supplementary Note 10 is a medical facility evaluation method including: an acquisition step of obtaining a quantitatively measured evaluation information about a medical facility that is a candidate of a transfer destination of a patient; and a calculation step of calculating a score indicating goodness of fit of the medical facility as the transfer destination with respect to the patient, on the basis of the evaluation information.

### (Supplementary Note 11)

A computer program described in Supplementary Note 11 is a computer program that allows a computer to execute: an acquisition step of obtaining a quantitatively measured evaluation information about a medical facility that is a candidate of a transfer destination of a patient; and a calculation step of calculating a score indicating goodness of fit of the medical facility as the transfer destination with respect to the patient, on the basis of the evaluation information.

### (Supplementary Note 12)

On a recording medium described in Supplementary Note 12, the computer program described in Supplementary Note 11 is recorded.

The present invention is not limited to the examples described above and is allowed to be changed, if desired, without departing from the essence or spirit of the invention which can be read from the claims and the entire specification. A medical facility evaluation apparatus, a medical facility evaluation method, and a computer program with such modifications are intended to be within the technical scope of the invention.

### DESCRIPTION OF REFERENCE CODES

1 Medical Facility Evaluation apparatus
11 CPU
12 RAM
13 ROM
14 Storage apparatus
15 Input apparatus
16 Output apparatus
17 Data bus
110 Evaluation information acquisition unit
111 User evaluation acquisition unit
112 Staff evaluation acquisition unit
113 Facility data evaluation acquisition unit
120 Score calculation unit
130 Transfer destination presentation unit

What is claimed is:

1. A medical facility evaluation apparatus comprising:
at least one memory that is configured to store informations; and
at least one processor that is configured to execute instructions
to obtain a quantitatively measured evaluation information about a medical facility that is a candidate of a transfer destination of a patient; and
to calculate a score indicating goodness of fit of the medical facility as the transfer destination with respect to the patient, on the basis of the evaluation information.

2. The medical facility evaluation apparatus according to claim 1, wherein the processor is further configured to execute instruction to present the medical facility to be recommended as the transfer destination of the patient on the basis of the score of each of a plurality of medical facilities.

3. The medical facility evaluation apparatus according to claim 2, wherein the processor presents a factor that causes a rise in the score of the medical facility to be recommended as the transfer destination of the patient.

4. The medical facility evaluation apparatus according to claim 1, wherein the evaluation information includes at least one of an evaluation by a user of the medical facility, an evaluation by an outsourcer staff of the medical facility, and an evaluation based on information about the medical facility.

5. The medical facility evaluation apparatus according to claim 4, wherein the evaluation information includes information about a time length required from when an inpatient of the medical facility calls a staff of the medical facility to when the staff of the medical facility responds.

6. The medical facility evaluation apparatus according to claim 4, wherein the evaluation information includes information about a cumulative time in which a staff of the medical facility responds to an inpatient of the medical facility.

7. The medical facility evaluation apparatus according to claim 4, wherein the evaluation information includes at least one of information about an activity amount, sleeping hours, and a conversation amount of an inpatient of the medical facility.

8. The medical facility evaluation apparatus according to claim 4, wherein the evaluation information includes information about a period in which an emotional state of an inpatient of the medical facility is positive.

9. The medical facility evaluation apparatus according to claim 1, wherein
the processor obtains a plurality of types of evaluation informations, and
the processor calculates the score by weighting each of the plurality of types of evaluation informations.

10. A medical facility evaluation method comprising:
obtaining a quantitatively measured evaluation information about a medical facility that is a candidate of a transfer destination of a patient; and
calculating a score indicating goodness of fit of the medical facility as the transfer destination with respect to the patient, on the basis of the evaluation information.

11. A non-transitory recording medium on which a computer program that allows a computer to execute a medical facility evaluation method is recorded, the medical facility evaluation method including:
obtaining a quantitatively measured evaluation information about a medical facility that is a candidate of a transfer destination of a patient; and
calculating a score indicating goodness of fit of the medical facility as the transfer destination with respect to the patient, on the basis of the evaluation information.
